(19) United States
(12) Patent Application Publication
(10) Pub. No.: US 2025/0302305 A1
(43) Pub. Date: Oct. 2, 2025

(54) Devices, Systems, and Methods for Mitigating Fluorescent Effect of an Optical Sensor

(71) Applicant: Google LLC, Mountain View, CA (US)

(72) Inventors: Hamed Vavadi, Castro Valley, CA (US); Peter Winthrop Richards, San Francisco, CA (US); Kaiyuan Yao, Fremont, CA (US); Herschel Max Watkins, Sausalito, CA (US)

(21) Appl. No.: 18/624,802

(22) Filed: Apr. 2, 2024

(51) Int. Cl. A61B 5/00

(52) U.S. Cl. CPC A61B 5/0071; A61B 5/443; A61B 5/681

(57) ABSTRACT

A computing device for measuring an intensity level of at least a first returned light signal is provided. The device includes an optical sensor and a processor. The optical sensor includes an emitter package defining a cavity, the cavity including a first die configured to output a first emitted light signal having a first wavelength and a second die configured to output a second emitted light signal having a second wavelength, wherein an optical isolation structure separates the first die from the second die within the cavity; and (ii) a first detector configured to receive the first returned light signal. Further, the processor is configured to determine the intensity level of the first returned light signal. A method of measuring an intensity level of at least a first returned light signal via the computing device is also provided.

### FIELD

The disclosure relates generally to any computing device that utilizes optical sensors to measure a parameter. In one specific example, the disclosure relates to wearable computing devices that utilize optical sensors to measure skin autofluorescence (SAF) via a diffuse optical method.

### BACKGROUND

Skin autofluorescence (SAF) can be used to non-invasively detect for the presence of and measure levels of advanced glycation end products (AGEs) that are present below a surface of a person's skin. AGEs are biomarkers that have been associated with aging and cardiovascular health and have also been implicated in such conditions as diabetes, atherosclerosis, kidney disease, and Alzheimer's disease. In particular, skin autofluorescence can be strongly correlated with health outcomes and could be used in conjunction with other physiologic data (e.g., heart rate and oxygen saturation (SpO2)) to provide information to a person about the person's health. Typically, a light source or emitter (e.g., a light emitting diode) and a light detector (e.g., a photodiode) are utilized in the devices that measure the intensity of returned light signals that are used to determine a level of skin autofluorescence, where it is understood that the fluorescence emission occurs at a different wavelength than the light source illumination. However, the accuracy of SAF measurements (or any type of measurement pertaining to light signals) can be impacted by stray light from the light emitter ultimately contaminating, tainting, or otherwise mixing with other light emitters (dies) present in the emitter package or system and/or the light detectors. In the particular case of measuring SAF in a system that utilizes multiple light emitters for various types of sensor applications, stray light from the SAF light emitter (e.g., an ultraviolet light emitting diode which is selected in order to initiate the fluorescent effect below a surface of skin) can strike another light emitter (e.g., a green light emitting diode used for emitting a light signal utilized in a photoplethysmography (PPG) sensor), resulting in the other light emitter emitting unwanted green light. The green light that is emitted potentially confounds the biological fluorescence signal that is returned to the light detector in response to the emitted ultraviolet light signal interacting with AGEs below the skin's surface, which is expected to have a wavelength similar to that of the green light associated with the PPG light emitter.

As such, a need exists for a device, system, and method of measuring the intensity of one or more returned light signals from a light emitter via a light detector in an accurate manner that minimizes or eliminates the contamination by other light signals associated with other light emitters and/or detectors. Such devices, systems, and methods would be useful in various combinations of light wavelengths over a broad range of applications in which important features of the signal being returned to a light detector from the skin are sensitive to the wavelength of the light emitted by the emitter in order to provide accurate measurements.

### SUMMARY

Aspects and advantages of embodiments of the disclosure will be set forth in part in the following description, or can be learned from the description, or can be learned through practice of the example embodiments.

In one aspect, a computing device for measuring an intensity level of at least a first returned light signal is provided. The device includes an optical sensor and a processor. The optical sensor includes an emitter package defining a cavity, the cavity including a first die configured to output a first emitted light signal having a first wavelength and a second die configured to output a second emitted light signal having a second wavelength, wherein an optical isolation structure separates the first die from the second die within the cavity; and (ii) a first detector configured to receive the first returned light signal. Further, the processor is configured to determine the intensity level of the first returned light signal. A method of measuring an intensity level of at least a first returned light signal via the computing device is also provided.

In some implementations, a filter coating can be disposed on an upper surface of the emitter package above the first die, the second die, or both.

In some implementations, the computing device can include control circuitry configured to reverse bias the second die while the first die is emitting the first emitted light signal and/or to reverse bias the first die while the second die is emitting the second emitted light signal.

In some implementations, the optical sensor can further include a second detector configured to receive at least a second returned light signal. In addition, an optical filter can be included that can block a portion of the first returned light signal from reaching the first detector, blocks a portion of the second returned light signal from reaching the second detector, or both. The optical filter can be a long pass filter that prevents light having a wavelength that is equal to the first wavelength from reaching the first detector, prevents light having a wavelength that is equal to the second wavelength from reaching the second detector, or both. However, it should be understood that other filters, such as short pass filters, are also contemplated by the present disclosure. Further, the processer can be configured to calculate, inter alia, a skin autofluorescence level based on a measured intensity level of the first returned light signal and a measured intensity level of the second returned light signal.

In some implementations, the first wavelength, the second wavelength, or both can range from about 250 nanometers to about 900 nanometers. For instance, the first wavelength, the second wavelength, or both can range from about 275 nanometers to about 500 nanometers.

In some implementations, a light blocking material can be disposed between the emitter package and the first detector, the second detector, or both.

In some implementations, the computing device can be a wearable computing device, and the optical sensor can be in contactor with a user's skin.

In another aspect, a method of measuring an intensity level of at least a first returned light signal via the computing device is also provided. The method includes providing an emitter package defining a cavity, the cavity including a first die configured to output a first emitted light signal having a first wavelength and a second die configured to output a second emitted light signal having a second wavelength, wherein an optical isolation structure separates the first die from the second die within the cavity; emitting, by the first die of the emitter package of an optical sensor of the computing device, the first emitted light signal; obtaining, by a first detector of the optical sensor of the computing device, the first returned light signal; and calculating, by a processor, the intensity level of the first returned light signal.

In some implementations, a filter coating can be disposed on an upper surface of the emitter package above the first die, the second die, or both.

In some implementations, the method further includes applying, via control circuitry, a reverse bias to the second die while the first die is emitting the first emitted light signal and/or to the first die while the second die is emitting the second emitted light signal.

In some implementations, the method can further include obtaining, by a second detector of the optical sensor of the computing device, at least a second returned light signal.

In some implementations, the method can further include blocking, via an optical filter, a portion of the first returned light signal from reaching the first detector, a portion of the second returned light signal from reaching the second detector, or both.

In some implementations, the optical filter can be a long pass filter that prevents light having a wavelength that is equal to the first wavelength from reaching the first detector, prevents light having a wavelength that is equal to the second wavelength from reaching the second detector, or both.

In some implementations, the method can further include calculating a skin autofluorescence level based on a measured intensity level of the first returned light signal and a measured intensity level of the second returned light signal.

In some implementations, the first wavelength, the second wavelength, or both can range from about 250 nanometers to about 900 nanometers. For instance, the first wavelength, the second wavelength, or both can range from about 275 nanometers to about 500 nanometers.

In some implementations, a light blocking material can be disposed between the emitter package and the first detector, the second detector, or both.

In some implementations, the computing device can be a wearable computing device, and the optical sensor can be in contactor with a user's skin.

These and other features, aspects, and advantages of various embodiments of the disclosure will become better understood with reference to the following description, drawings, and appended claims. The accompanying drawings, which are incorporated in and constitute a part of this specification, illustrate example embodiments of the disclosure and, together with the description, serve to explain the related principles.

### BRIEF DESCRIPTION OF THE DRAWINGS

Detailed discussion of example embodiments directed to one of ordinary skill in the art is set forth in the specification, which makes reference to the appended drawings, in which:

FIG. 1 depicts a perspective front view of an example computing device (e.g., a wearable computing device) according to some implementations of the present disclosure.

FIG. 2 depicts a rear view of an example computing device according to some implementations of the present disclosure.

FIG. 3 is a cross-sectional schematic illustration of a portion of an optical sensor of the computing device according to one embodiment of the disclosure when the optical sensor is placed in direct contact with a user's skin, particularly showing the resulting optical path (e.g., emitted light signal and detected light signal) used to measure skin autofluorescence.

FIG. 4 is a perspective view of one embodiment of a light emitter package according to some implementations of the present disclosure.

FIG. 5 is a top view of the light emitter package of FIG. 4.

FIG. 6 is a top view of one embodiment of a light emitter package according to some implementations of the present disclosure.

FIG. 7 is a top view of one embodiment of a light emitter package according to some implementations of the present disclosure.

FIG. 8 is a top view of one embodiment of a light emitter package according to some implementations of the present disclosure.

FIG. 9 is a top view of one embodiment of a light emitter package according to some implementations of the present disclosure.

FIG. 10 is a top view of one embodiment of a light emitter package according to some implementations of the present disclosure.

FIG. 11 is a top view of one embodiment of a light emitter package according to some implementations of the present disclosure.

FIG. 12 is a top view of one embodiment of a light emitter package according to some implementations of the present disclosure.

FIG. 13 is a top view of one embodiment of a light emitter package according to some implementations of the present disclosure.

FIG. 14 is a top view of one embodiment of a light emitter package according to some implementations of the present disclosure.

FIG. 15 is a top view of one embodiment of a light emitter package according to some implementations of the present disclosure.

FIG. 16 is a top view of one embodiment of a light emitter package according to some implementations of the present disclosure.

FIG. 17 is a top view of one embodiment of a light emitter package according to some implementations of the present disclosure.

FIG. 18 depicts an example block diagram of an example computing device according to some implementations of the present disclosure.

FIG. 19 illustrates a flow diagram for a method for measuring skin autofluorescence using a wearable computing device.

Reference numerals that are repeated across plural figures are intended to identify the same features in various implementations.

### DETAILED DESCRIPTION

Reference now will be made to embodiments of the disclosure, one or more examples of which are illustrated in the drawings. Each example is provided by way of explanation of the disclosure and is not intended to limit the disclosure. In fact, it will be apparent to those skilled in the art that various modifications and variations can be made to disclosure without departing from the scope or spirit of the disclosure. For instance, features illustrated or described as part of one embodiment can be used with another embodiment to yield a still further embodiment. Thus, it is intended that the disclosure covers such modifications and variations as come within the scope of the appended claims and their equivalents.

Terms used herein are used to describe the example embodiments and are not intended to limit and/or restrict the disclosure. The singular forms “a,” “an” and “the” are intended to include the plural forms as well, unless the context clearly indicates otherwise. In this disclosure, terms such as “including”, “having”, “comprising”, and the like are used to specify features, numbers, steps, operations, elements, components, or combinations thereof, but do not preclude the presence or addition of one or more of the features, elements, steps, operations, components, or combinations thereof.

It will be understood that, although the terms first, second, third, etc., may be used herein to describe various elements, the elements are not limited by these terms. Instead, these terms are used to distinguish one element from another element. For example, without departing from the scope of the disclosure, a first element may be termed as a second element, and a second element may be termed as a first element.

The term “and/or” includes a combination of a plurality of related listed items or any item of the plurality of related listed items. For example, the scope of the expression or phrase “A and/or B” includes the item “A”, the item “B”, and the combination of items “A and B”.

In addition, the scope of the expression or phrase “at least one of A or B” is intended to include all of the following: (1) at least one of A, (2) at least one of B, and (3) at least one of A and at least one of B. Likewise, the scope of the expression or phrase “at least one of A, B, or C” is intended to include all of the following: (1) at least one of A, (2) at least one of B, (3) at least one of C, (4) at least one of A and at least one of B, (5) at least one of A and at least one of C, (6) at least one of B and at least one of C, and (7) at least one of A, at least one of B, and at least one of C.

Generally speaking, the present disclosure is directed to a computing device, system, and method of use of a computing device for measuring an intensity of one or more returned light signals with reduced contamination from one or more light emitters and/or one or more light detectors. The device includes an optical sensor that includes a light source or emitter package (e.g., a light emitting diode array). The emitter package includes a first die configured to output a first emitted light signal having a first wavelength and a second die configured to output a second emitted light signal having a second wavelength, although it is to be understood that additional dies, such as a third die, fourth die, fifth die, and so on are also contemplated by the present disclosure. The first die, the second die, and any other dies present are located within a cavity defined by the emitter package. Further, an internal optical isolation structure separates the first die from the second die and/or any other dies present within the cavity. Without intending to be limited by any particular theory, the present inventors have found that the internal optical isolation structure contained within the cavity of the emitter package can isolate the dies from each other and limit any contamination caused by, for instance, light from the first emitted light signal associated with the first die from striking the second die and causing it to emit light and vice versa. The device also includes at least a first detector configured to receive at least a first returned light signal and a processor configured to determine an intensity level of the first returned light signal.

Additionally or alternatively, a filter coating can be present on an upper surface of the light emitter package, and the filter coating can be disposed on the upper surface above the first die, the second die, any other dies present, or a combination thereof. The filter coating above each die can block certain wavelengths of an emitted light signal while allowing other wavelengths of the emitted light signal for each respective die to pass through the filter coating. The present inventors have found that the application of filter coatings can fine tune the wavelength range of each of the emitted light signals to create a specific emitted light signal that can be tailored for a specific application, such as preventing the initiation of a fluorescent effect from one die when another die is emitting a light signal of a particular wavelength, such as in the case of measuring skin autofluorescence.

Additionally or alternatively, the computing device can also include control circuitry that can reverse bias the second die while the first die is emitting the first emitted light signal, which can reduce the emission of light from the second die and thus reduce contamination from the second die. Likewise, the computing device can also include control circuitry that can reverse bias the first die while the second die is emitting the second emitted light signal, which can reduce the emission of light from the first die and thus reduce contamination from the first die. Further, it should be understood that the control circuitry can be configured to reverse bias any of the dies contained within the light emitter package while any of the other dies are emitting an emitted light signal.

The above features, either alone or in combination, create a higher level of spectral purity than is typically achieved using the types of optical sensors used in wearable computing devices, but it should be understood that the present disclosure contemplates utilizing such features in a wide array of applications including bio-imaging, security, lighting, optoelectronics, etc.

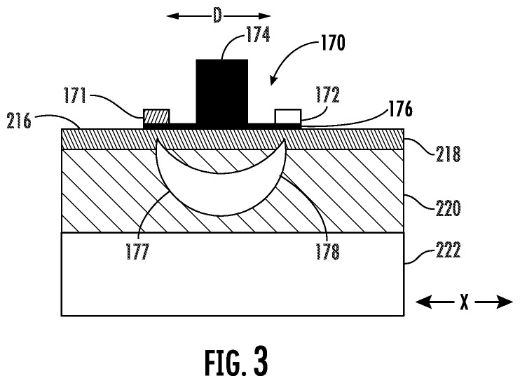

Further, it should be understood that the devices contemplated by the present disclosure utilize a diffuse optical geometry where the light emitter and the light detector are separated from each other laterally by a sufficient distance and are also placed in close proximity, or in contact with, the user's skin (see FIG. 3). Moreover, light blocking materials or structures can also be disposed between the emitter package and light detector to minimize specular reflection and the reflection of light directly off the skin's surface and into the light detector. As such, light is forced to dive into the user's tissue with a banana-shaped path trajectory and at a deep penetration depth. Since a majority of skin autofluorescence signal originates from collagen cross-linking, and collagen is most abundant in the subsurface dermis layer beneath the epidermis located at the surface of the user's skin, it follows that the diffuse geometry contemplated by the devices of the present disclosure provide a strong skin autofluorescence signal compared to the existing reflective geometry that is known in the prior art. In addition, the devices and methods of the present disclosure contemplate that the skin autofluorescence measurements can be taken continuously while the user is wearing the wearable computing device, which can improve accuracy of skin autofluorescence measurements compared to existing AGE readers. For instance, the devices and methods of the present disclosure contemplate taking measurements continuously while the device is being worn, which includes taking intermittent measurements throughout the day that can be spaced apart by a time frame of about 0.0001 seconds to about 24 hours, or any range therebetween, as opposed to taking one discrete measurements as is done with existing AGE readers.

Further, in one particular implementation, the present disclosure contemplates an optical sensor arrangement for measuring skin autofluorescence that utilizes at least one light emitter that is one of the dies in an emitter package and at least two light detectors, where one light detector includes a long pass filter that blocks the non-fluorescent portion of the returned light signal in order to only focus on measuring the intensity of the fluorescent portion of the returned light signal, which further improves the accuracy of the skin autofluorescence readings. For instance, the optical long pass filter can prevent light having a wavelength of less than about 450 nanometers, such as UV or near-UV light, from reaching the detector that includes the long pass filter.

Example aspects of the present disclosure are directed to a wearable computing device that can be worn, for example, on a user's wrist. The wearable computing device includes an optical sensor that can be configured to generate a returned light signal that is indicative of a biometric (e.g., skin autofluorescence level) of the user. The optical sensor includes one or more light emitters as part of an emitter package that can include one or more light sources (e.g., light emitting diodes (LEDs)) configured to emit a light signal toward a body part of the user when the wearable computing device is worn by the user. The optical sensor can further include two or more detectors (e.g., photodiodes) configured to receive a reflection of the light emitted toward the body part. The ratio of these two returned light signals can then be used to determine a skin autofluorescence level of the user, which can then be used to determine various health metrics associated with, but not limited to, cardiovascular health, diabetes, atherosclerosis, kidney disease, and Alzheimer's disease.

The optical sensors of the wearable computing device can also include a PPG sensor that is configured to generate a PPG signal indicative of a biometric (e.g., heart rate) of the user. The PPG sensor includes one or more light emitters that can include one or more light sources (e.g., light emitting diodes (LEDs)) configured to emit light toward a body part of the user when the wearable computing device is worn by the user. The PPG sensor further includes one or more detectors (e.g., photodiodes) configured to receive a reflection of the light emitted toward the body part. It should be understood that the PPG signal is the reflection of the light.

Moreover, it should be understood that the optical sensor and emitter package features of the present disclosure are not limited to improving spectral purity in SAF applications where ultraviolet or near-ultraviolet light is emitted to then initiate a fluorescent effect below the skin's surface and can be used to improve the spectral purity, and hence light intensity measurements, of any returned light signals of interest in any application where interference or cross-talk between dies in an emitter package can occur.

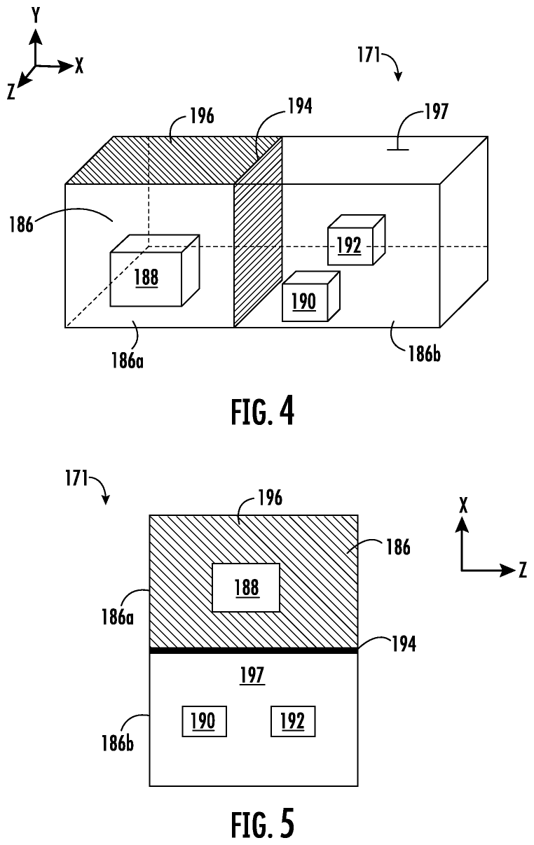

Referring now to the drawings, FIGS. 1 through 3 illustrate examples of a computing device 100 according to various examples of the present disclosure, while FIGS. 4-17 focus more specifically on the emitter package 171 that is part of the computing device 100 contemplated by the present disclosure. The computing device 100 can be in the form of a wearable computing device that can be worn, for example, on a body part 102 (e.g., an arm, wrist, etc.) of a user. The computing device 100 includes a body 110 having an outer facing surface 165, which can be referred to as the front of the wearable computing device 100, and a skin contacting surface 166, which can be referred to as the back of the wearable computing device 100. Furthermore, the body 110 defines a cavity (not shown) between the outer facing surface 165 and the skin contacting surface 166 in which one or more electronic components (e.g., disposed on one or more printed circuit boards) are disposed. The computing device 100 includes a printed circuit board (not shown) disposed within the cavity. Furthermore, one or more electronic components are disposed on the printed circuit board. The computing device 100 can further include a battery that is disposed within the cavity defined by the body 110.

In FIG. 1 the computing device 100 includes a first band 130 and a second band 132. As shown, the first band 130 is coupled to the body 110 at a first location thereon. Conversely, the second band 132 is coupled to the body 110 at a second location thereon. Furthermore, the first band 130 and the second band 132 can be coupled to one another to secure the body 110 to the body part 102 of the user.

In some examples, the first band 130 can include a buckle or clasp (not shown). Additionally, the second band 132 can include a plurality of apertures (not shown) spaced apart from one another along a length of the second band 132. In such implementations, a prong of the buckle associated with the first band 130 can extend through one of the plurality of openings defined by the second band 132 to couple the first band 130 to the second band 132. It should be appreciated that the first band 130 can be coupled to the second band 132 using any suitable type of fastener. For example, in an embodiment, the first band 130 and the second band 132 can include a magnet. In such implementations, the first band 130 and the second band 132 can be magnetically coupled to one another to secure the body 110 to a body part 102 (e.g., an arm) of the user.

In FIG. 1, the computing device 100 includes a cover 140 positioned on the body 110 so that the cover 140 is positioned on top of a display 182. In this manner, the cover 140 can protect the display 182 from being scratched. In an embodiment, the computing device 100 can include a seal (not shown) positioned between the body 110 and the cover 140. For instance, a first surface of the seal can contact the body 110 and a second surface of the seal can contact the cover 140. In this manner, the seal between the body 110 and the cover 140 can prevent a liquid (e.g., water) from entering the cavity defined by the body 110.

It should be understood that the cover 140 can be optically transparent so that the user can view information being displayed on the display 182. For instance, in an embodiment, the cover 140 can include a glass material. It should be understood, however, that the cover 140 can include any suitable optically transparent material.

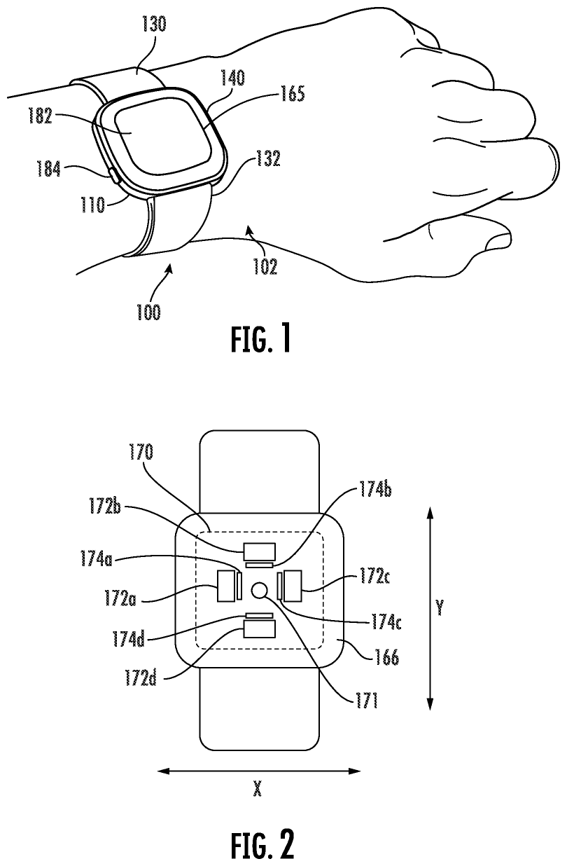

Referring to FIG. 2, the computing device 100 further includes various sensors 170 (e.g., optical sensors) that are disposed within the cavity defined by the body 110 or on a surface of the body 110. For example, an optical sensor 170 may include one or more skin autofluorescence sensors and/or one or more photoplethysmography (PPG) sensors disposed on a skin contacting surface 166 of the body 110. The skin autofluorescence sensors can, for example, be used to monitor for advanced glycation end products below a surface of the user's skin. The optical sensor 170 can include one or more light source or emitter packages 171 (e.g., an array light-emitting diodes (LEDs) in the form of dies contained within a cavity) and one or more light detectors 172a-d (e.g., photodiodes). Meanwhile, the PPG sensor(s) can, for example, be used to monitor a heart rate of the user. The PPG sensor(s) can also include one or more light source or emitter packages 171 (e.g., an array of light-emitting diodes (LEDs) in the form of dies contained within a cavity) and one or more light detectors (e.g., photodiodes) 172a-d. Further, it is also to be understood that the same emitter package 171 and light detector(s) 172 (see FIG. 3) can be used as both the SAF sensor and the PPG sensor depending on which die within the emitter package 171 is emitting an emitted light signal at a given time.

In FIG. 2, a skin contacting surface 166 (e.g., a rear surface) of an example wearable computing device 100 is illustrated according to one or more example embodiments of the disclosure. It should be understood that the although only one light source or emitter package 171 is shown, multiple emitter packages 171 can be utilized, where one light source or emitter package 171 can be associated with the skin autofluorescence sensor portion of the optical sensor 170 and another light source or emitter package (not shown) can be associated with the PPG sensor portion of the optical sensor 170. In particular, the one or more light sources or emitter packages 171 can include dies that can emit light signals having a wavelength ranging from about 250 nanometers to about 900 nanometers, such as from about 275 nanometers to about 700 nanometers, such as from about 300 nanometers to about 600 nanometers. In one particular embodiment, the emitter package 171 for the skin autofluorescence sensor portion of the optical sensor 170 can be a near-ultraviolet LED die, meaning the die emits light having a wavelength ranging from about 350 nanometers to about 450 nanometers.

Referring still to FIG. 2, the two or more light detectors, which can be used for measuring skin autofluorescence with the optical sensor 170, can be selected any combination of detectors 172a, 172b, 172c, and/or 172d, so long as a light blocking material 174a, 174b, 174c, and/or 174d is disposed between the emitter package 171 and any combination of the detectors 172a, 172b, 172c, and/or 172d that are utilized for the skin autofluorescence sensor portion of the optical sensor 170. The light blocking material 174a, 174b, 174c, and/or 174d can be made of any suitable material that prevents the light emitted from the emitter 171 and reflected off the surface of the user's skin 216 from reaching any of the detectors 172a, 172b, 172c, and/or 172d, which could affect the accuracy of the skin autofluorescence measurements by the optical sensor 170. For instance, the light blocking material 174a, 174b, 174c, and/or 174d can be an opaque material, such as an opaque plastic or composite material. Further, the emitter package 171 and any of the detectors 172a, 172b, 172c, and/or 172d utilized in the skin autofluorescence portion of the optical sensor 170 can be spaced apart from each other by a distance D ranging from about 0.5 millimeters to about 6 millimeters, such as from about 0.75 millimeters to about 5 millimeters, such as from about 1 millimeter to about 4 millimeters in the X (horizontal) direction or Y (vertical) direction, where the distance is measured from a edge of the emitter package 171 to an edge of any one of the detectors 172, as shown in FIG. 3. Furthermore, more than one light source or emitter package 171 (e.g., an array of LEDs) containing a plurality of LED dies may be included such that different detectors may be combined with different emitter packages 171 and/or each detector 172 may be combined with one or more emitter packages 171 to output a respective skin autofluorescence.

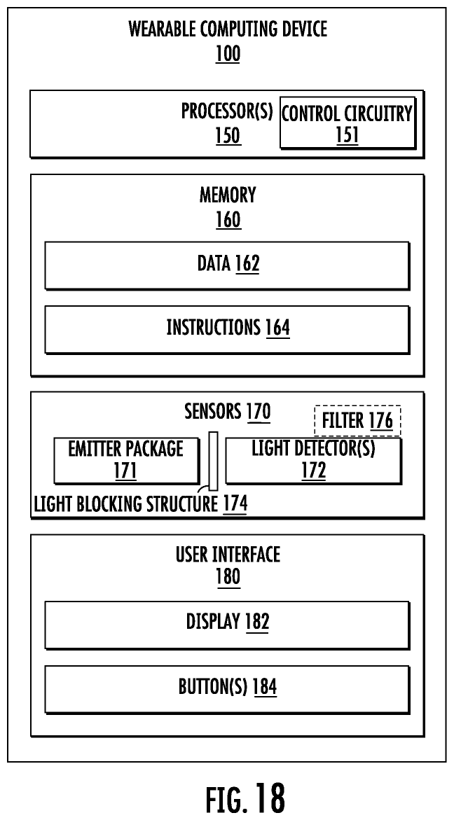

In addition, assuming, for example, that the autofluorescence portion of the optical sensor 170 utilizes a die from emitter package 171 and detectors 172a and 172b, one of the detectors 172a can include an external optical filter 176 (see FIG. 18) that is an optical long pass filter. The optical long pass filter 176 can, in some embodiments, prevent ultraviolet light (e.g., a light signal having a wavelength of less than about 500 nanometers, such as less than about 450 nanometers, such as less than about 425 nanometers, such as less than about 400 nanometers) from reaching detector 172a, although it is to be understood that the optical long pass filter 176 can, in other embodiments, alternatively be used to block light having a wavelength that is equal to the wavelength of the emitted light signal 177, regardless of what that wavelength is, from reaching detector 172a. In some embodiments, the external optical filter 176 can be formed by coating a filtering material onto a silicon photodiode, where such coating materials can include silicon dioxide, zinc oxide, polycarbonate, and combinations thereof. In this manner, in an embodiment where the emitter package 171 includes a die that is an ultraviolet light emitting diode, detector 172a may only detect light having a wavelength of greater than 400 nanometers, such as greater than 425 nanometers, such as greater than about 450 nanometers, such as greater than about 500 nanometers, where such light is associated with the fluorescent portion of the light signal's wavelength spectrum that is of interest in measuring skin autofluorescence. In other words, the external optical long pass filter 176 can prevent light having a wavelength that is equal to the wavelength of the one or more emitted light signals 177 emitted by the one or more emitters 171 from reaching detector 172a.

Referring to FIG. 3, more details about such an arrangement are described further. In particular, FIG. 3 is a cross-sectional schematic illustration of a portion of the autofluorescence portion of the optical sensor of the wearable computing device according to one embodiment of the disclosure when the sensor is placed in direct contact with a surface of a user's skin 216, particularly showing the resulting optical path (e.g., emitted light signal 177 and returned light signal 178) from a light source or emitter 171 to a light detector 172 used to measure skin autofluorescence, where a light blocking material 174 prevents the emitted light signal 177 from overlapping with the detected light signal 178. As shown, the optical path is able to penetrate below the epidermis 218 at the skin's surface 216 to the dermis 220. Further, although not shown, it is contemplated that at least a portion of the optical path can also reach the subcutaneous tissue 222. The wearable device 100 of the present disclosure and illustrated in FIG. 3 employs a diffusive optical geometry where the light source or emitter 171 and the light detector 172 are laterally separated by a distance D that can range up to about several millimeters in the X-direction and are placed in close proximity to users' skin 216 as discussed in detail above. Light blocking materials 174 are also implemented to minimize specular reflection. Thus, the light emitted from the light source is forced to dive into the tissue beneath the skin with a banana-shaped trajectory and deep penetration depth.

Further, it should be noted that a diffuse reflectance geometry as contemplated by the present disclosure increases the signal and provides a more accurate SAF reading compared to a purely reflective geometry as known in the prior art. More specifically, the current standard for measuring skin autofluorescence measures the intensity of the fluoresced light at a detector at a distance of several centimeters from the skin, and normalizes this to the intensity of reflectance light at a point similarly distant from the skin. In contrast, the wearable device of the present disclosure for SAF measurement uses a diffuse optical design. This is an important differentiator from existing designs, as fluorescence is inherently diffuse. This is because during fluorescence the direction of the emission photon is independent of the direction of the excitation photon. By normalizing the fluorescence to the correct type of reflectance, noise caused by variation in specular reflectivity (glossiness or shininess) is eliminated, reducing error. Further measured light has necessarily traveled through the skin, increasing the fraction of light which will be absorbed and subsequently fluoresced by the skin.

Still referring to FIG. 3, when an emitted light signal 177 is emitted from an LED die that is part of the emitter package 171, which can be in direct contact with the user's surface of skin 216 or spaced apart from the user's surface of skin 216 by a distance of from about 0 millimeters to about 0.5 millimeters in the event that there is a small gap due to loss of contact during movement of the wearable computing device, the user's skin absorbs most of the emitted light signal 177 and reflects some of the emitted light signal 177, while some of the emitted light signal 177 is shifted to a longer wavelength by advanced glycation end products (AGEs) below the epidermis 218, resulting in a returned light signal 178 that has, inter alia, a fluorescent component. The average distance by which the emitted light signal 177 can penetrate beneath the user's surface of skin 216 can range from about 0.01 millimeters to about 3 millimeters, such as from about 0.05 millimeters to about 2 millimeters, such as from about 0.1 millimeters to about 2.5 millimeters. An intensity level of all components of the returned light signal 178 is what would normally be measured or determined by the detector 172a, but to obtain an accurate reading that focuses on the fluorescent component only, which is correlated to the level of AGEs present below the skin at the dermis 220 and/or subcutaneous tissue 222 layers of the skin, the long pass filter 176 is utilized to block out non-fluoresced light so that only an intensity level of the fluoresced portions of the returned light signal 178 is determined. Meanwhile the other detector 172b receives the full spectrum of wavelengths from its returned light signal since no filter is utilized with the detector 172 and determines an intensity level of the returned light signal 178. From the intensity level measurements obtained by the detectors 172a and 172b, a ratio of what portion of the light signal's intensity is associated with the fluorescent component, and hence AGEs, can be calculated via one or more processors associated with the wearable computing device 100 to determine a level of skin autofluorescence present below a surface of the user's skin. This level can then be correlated to a level of AGEs present, which can be used to determine various health risks or conditions as described above.

In addition to the optical sensor 170 including a skin autofluorescence portion, the optical sensor 170 can also include a PPG portion that can include one or more PPG sensors. Each PPG sensor may correspond to a combination of one or more light sources or emitters 171 and one or more detectors 172a, 172b, 172c, and/or 172d. For example, the wearable computing device may include two or more PPG sensors. Furthermore, more than one light source or emitter package 171 containing multiple LED dies may be included such that different detectors 172a, 172b, 172c, and/or 172d may be combined with different LED dies and/or each detector may be combined with one or more LED dies to output a respective PPG signal.

It should be understood that the various light sources or emitter packages 171 and/or the various light detectors 172a, 172b, 172c, and/or 172d may be used in obtaining skin autofluorescence readings, PPG readings, or both and that the skin autofluorescence and/or PPG readings can be measured or determined continuously. Further, it should be understood that the present disclosure contemplates the measurement of other parameters associated with optical signals other than SAF or PPG signal intensity levels. Moreover, it should be understood that although only one light source or emitter package 171 is shown, multiple light sources or emitter packages 171 are contemplated by the present disclosure. In addition, the arrangement of the various light sources or emitter packages 171 and detectors 172, 172a, 172b, 172c, and/or 172d is not limited to the arrangement shown in FIG. 2. For example, in FIG. 2, the plurality of detectors may be spaced apart from each other at regular or irregular intervals. In FIG. 2, the emitter package 171 is disposed in a central portion of the optical sensor 170. Detectors 172a and 172c are spaced apart from the emitter package 171 and the light detectors 172b and 172d along the X-direction, and the detectors 172b and 172 are spaced about from the emitter package 171 and the detectors 172a and 172c along the Y-direction. However, the configuration of the detectors and emitter package may be different from that illustrated in FIG. 2, and the disclosure is not limited to the example of FIG. 2.

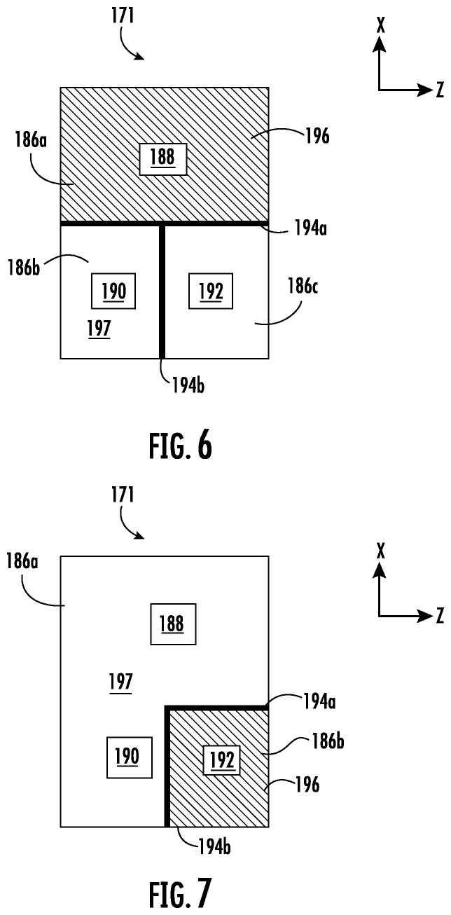

Turning now to FIGS. 4-17, the features of the emitter package 171 contemplated by the present disclosure and that allow for a higher level of spectral purity with respect to the light signals received by one or more light detectors 172 are described in more detail. In particular, FIG. 4 is a perspective view of one embodiment of an emitter package 171 according to some implementations of the present disclosure, while FIG. 5 is a top view of the emitter package of FIG. 5, while FIGS. 6-17 are top view of various other implementations of the emitter package 171 that are contemplated by the present disclosure. In particular, each of the implementations can include an emitter package 171 that defines a cavity 186 and has an upper surface 197. In FIGS. 4-17, the emitter package 171 includes a first die 188 located within a first portion 186a of the cavity 186 and (i) a second die 190 and a third die 192 located within a second portion 186b of the cavity 186, or (ii) a second die 190 located within a second portion 186b of the cavity and a third die 192 located within a third portion 186c of the cavity 186, although the present disclosure also contemplates the use of more than three dies. The first die 188 can be a green LED, the second die 190 can be an ultraviolet (UV) LED, and the third die 192 can be a red LED, although the present disclosure also contemplates any other combination of LEDs within the emitter package 171, where each of the dies is configured to emit a light signal having a particular wavelength. Further, an optical isolation structure 194 can separate the first die 188 from one or both of the second die 190 and the third die 192 within the cavity 186 such that the optical isolation structure 194 is internal to the emitter package 171. For example, the optical isolation structure 194 can isolate a green LED die from a UV LED die so that the light emitted from the UV LED die does not reach the green LED die to cause the green LED die to exhibit a fluorescent effect, which could affect the accuracy of any detectors measuring an intensity level of a returned light signal focusing on fluorescent light, as would be the case in SAF measurements. The optical isolation structure 194 can be any opaque material, such as an opaque plastic or composite material, that blocks light of various wavelengths.

Moreover, the emitter package 171 can include one or more filter coatings 196 disposed on an upper surface 197 of the emitter package 171. The filter coating 196 can function as a long pass filter, a short pass filter, a bandpass filter, or any other filter that blocks or reflects certain wavelengths of an emitted light signal and allows other wavelengths of the emitted light signal to pass through the emitter package 171.

The filter coating 196 can, in some embodiments, be a long pass filter and can be coated onto the upper surface 197 of the emitter package above the first portion 186a of the cavity 186 that can house a die 188 that is a green LED die to prevent any ultraviolet light (e.g., a light signal having a wavelength of less than about 500 nanometers, such as less than about 450 nanometers, such as less than about 425 nanometers, such as less than about 400 nanometers) from the ultraviolet LED, which can be die 190, from reaching the green LED die (in this case die 188), which could result in the green LED initiating a fluorescent effect that could reach one of the light detectors 172 and taint a returned light signal from, for instance, the ultraviolet LED die 190. However, it is to be understood that the filter coating 196 can, in other embodiments, alternatively be used to block or reflect an emitted light signal from one die having any predefined wavelength from reaching any of the other dies within the emitter package depending on the particular parameter being measured and/or the particular wavelength of the light signal that is intended to be emitted from the one die. Such filtering prevents tainting or contamination of the various emitted light signals from the various dies within the emitter package and improves spectral purity, resulting in more accurate intensity readings. In some embodiments, the external optical filter 176 can be formed by coating a filtering material mixed with clear epoxy onto the upper surface 197 of the emitter package 171, where such coating materials can include silicon dioxide, zinc oxide, polycarbonate, and combinations thereof.

In FIGS. 4-5 specifically, the emitter package 171 includes an internal optical isolation structure 194 that separates the first die 188 from the second die 190 and third die 192. In addition, a filter coating 196 is disposed on an upper surface 197 of the emitter package 171 above the first portion 186a of the cavity 186 where only the first die 188 is located, while second die 190 and third die 192 are located in a second portion 186b of the cavity 186.

In FIG. 6, the emitter package 171 includes an internal optical isolation structure 194 having a first portion 194a that separates the first die 188 from the second die 190 and third die 192, as well as a second portion 194b that separates the second die 190 from the third die 192, such that the first die 188 is located in a first portion 186a of the cavity, the second die 190 is located in a second portion 186b of the cavity, and the third die 192 is located in a third portion 186c of the cavity 186. In addition, a filter coating 196 is disposed on an upper surface 197 of the emitter package 171 above the first cavity portion 186a only.

In FIG. 7, the emitter package 171 includes an internal optical isolation structure 194 having a first portion 194a and a second portion 194b such that the third die 192, located in a second portion 186b of the cavity 186, is separated from the first die 188 and the second die 190, which are located in a first portion 186a of the cavity 186. In addition, a filter coating 196 is disposed on an upper surface 197 of the emitter package 171 above the third die 192 only.

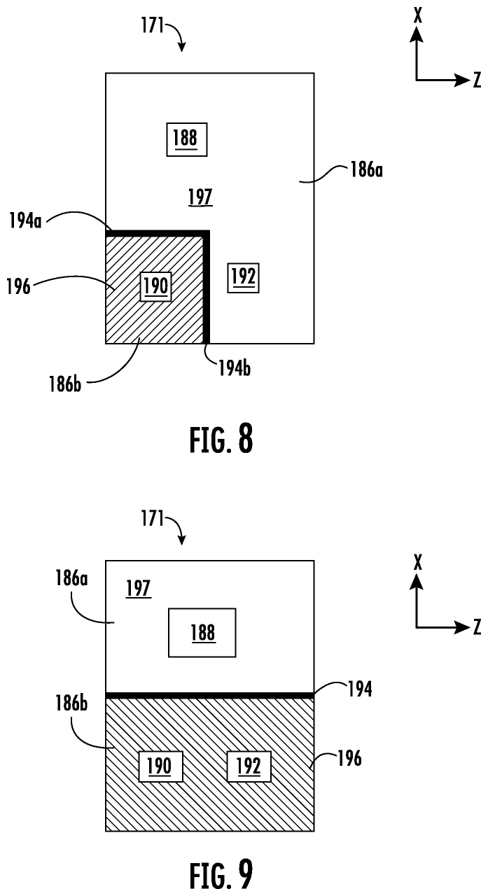

In FIG. 8, the emitter package 171 includes an internal optical isolation structure 194 having a first portion 194a and a second portion 194b such that the second die 190, located in a second portion 186b of the cavity 186, is separated from the first die 188 and the third die 192, which are located in a first portion 186a of the cavity 186. In addition, a filter coating 196 is disposed on an upper surface 197 of the emitter package 171 above the second die 190 only.

In FIG. 9, the emitter package 171 includes an internal optical isolation structure 194 that separates the first die 188 from the second die 190 and third die 192. In addition, a filter coating 196 is disposed on an upper surface 197 of the emitter package 171 above the second portion 186b of the cavity 186 where the second die 190 and the third die 192 are located, while the first die 188 is located in a first portion 186a of the cavity 186.

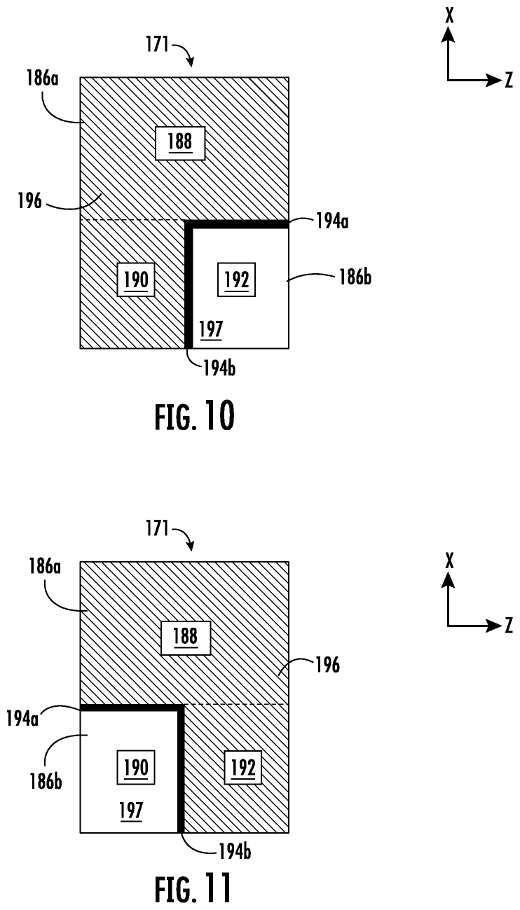

In FIG. 10, the emitter package 171 includes an internal optical isolation structure 194 having a first portion 194a and a second portion 194b such that the third die 192, located in a second portion 186b of the cavity 186, is separated from the first die 188 and the second die 190, which are located in a first portion 186a of the cavity 186. In addition, a filter coating 196 is disposed on an upper surface 197 of the emitter package 171 above the first die 188 and the second die 190.

In FIG. 11, the emitter package 171 includes an internal optical isolation structure 194 having a first portion 194a and a second portion 194b such that the second die 190, located in a second portion 186b of the cavity 186, is separated from the first die 188 and the third die 192, which are located in a first portion 186a of the cavity 186. In addition, a filter coating 196 is disposed on an upper surface 197 of the emitter package 171 above the first die 188 and the third die 192.

In FIG. 12, the emitter package 171 includes an internal optical isolation structure 194 having a first portion 194a that separates the first die 188 from the second die 190 and third die 192, as well as a second portion 194b that separates the second die 190 from the third die 192, such that the first die 188 is located in a first portion 186a of the cavity, the second die 190 is located in a second portion 186b of the cavity, and the third die 192 is located in a third portion 186c of the cavity 186. In addition, a first filter coating 196a is disposed on an upper surface 197 of the emitter package 171 above the second cavity portion 186b and a second filter coating 196b is disposed on an upper surface 197 of the emitter package 171 above the third cavity portion 186c.

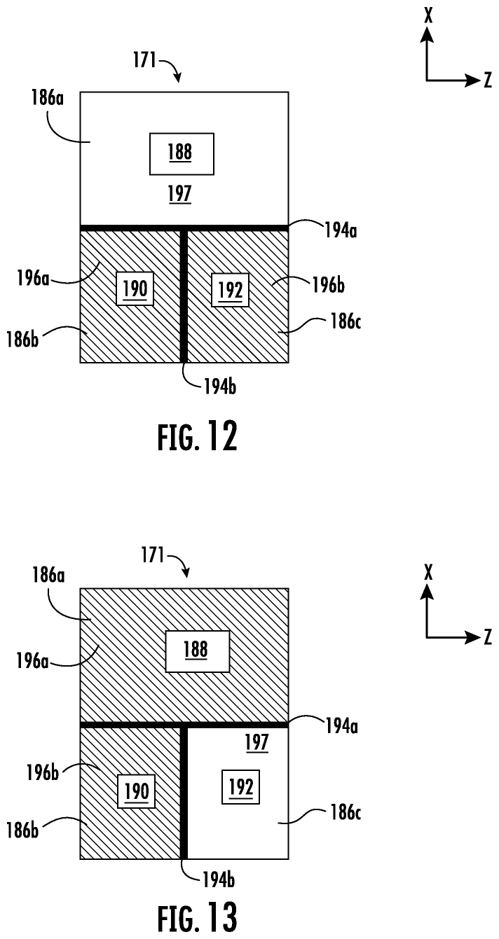

In FIG. 13, the emitter package 171 includes an internal optical isolation structure 194 having a first portion 194a that separates the first die 188 from the second die 190 and third die 192, as well as a second portion 194b that separates the second die 190 from the third die 192, such that the first die 188 is located in a first portion 186a of the cavity, the second die 190 is located in a second portion 186b of the cavity, and the third die 192 is located in a third portion 186c of the cavity 186. In addition, a first filter coating 196a is disposed on an upper surface 197 of the emitter package 171 above the first cavity portion 186a and a second filter coating 196b is disposed on an upper surface 197 of the emitter package 171 above the second cavity portion 186b.

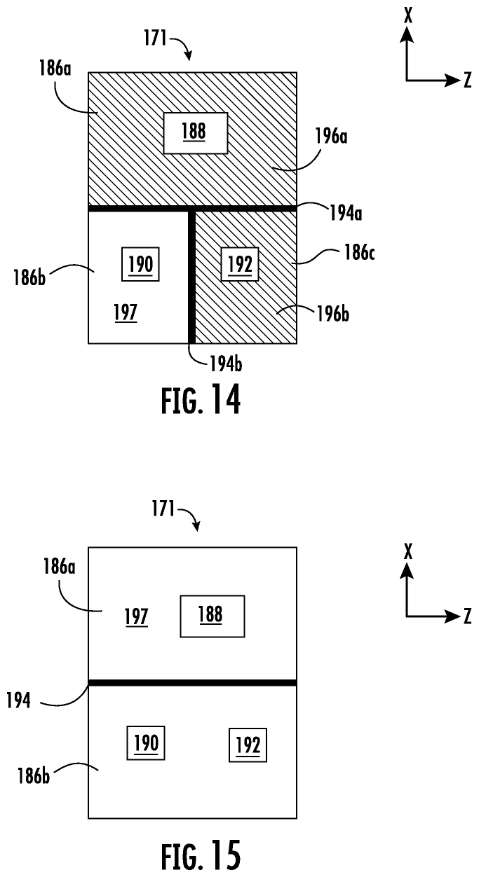

In FIG. 14, the emitter package 171 includes an internal optical isolation structure 194 having a first portion 194a that separates the first die 188 from the second die 190 and third die 192, as well as a second portion 194b that separates the second die 190 from the third die 192, such that the first die 188 is located in a first portion 186a of the cavity, the second die 190 is located in a second portion 186b of the cavity, and the third die 192 is located in a third portion 186c of the cavity 186. In addition, a first filter coating 196a is disposed on an upper surface 197 of the emitter package 171 above the first cavity portion 186a and a second filter coating 196b is disposed on an upper surface 197 of the emitter package 171 above the third cavity portion 186c.

In FIG. 15, the emitter package 171 includes an internal optical isolation structure 194 that separates the first die 188 from the second die 190 and third die 192. However, there is no filter coating 196 present.

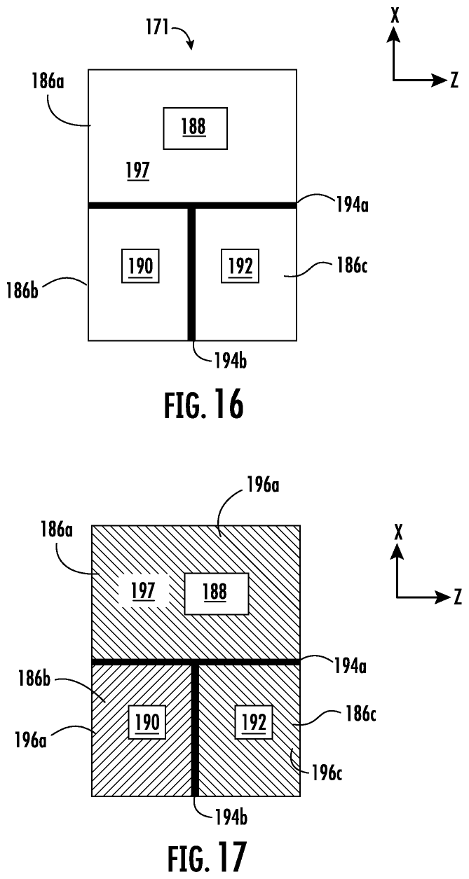

In FIG. 16, the emitter package 171 includes an internal optical isolation structure 194 having a first portion 194a that separates the first die 188 from the second die 190 and third die 192, as well as a second portion 194b that separates the second die 190 from the third die 192, such that the first die 188 is located in a first portion 186a of the cavity, the second die 190 is located in a second portion 186b of the cavity, and the third die 192 is located in a third portion 186c of the cavity 186. However, there is no filter coating 196 present.

In FIG. 17, the emitter package 171 includes an internal optical isolation structure 194 having a first portion 194a that separates the first die 188 from the second die 190 and third die 192, as well as a second portion 194b that separates the second die 190 from the third die 192, such that the first die 188 is located in a first portion 186a of the cavity, the second die 190 is located in a second portion 186b of the cavity, and the third die 192 is located in a third portion 186c of the cavity 186. In addition, a first filter coating 196a is disposed on an upper surface 197 of the emitter package 171 above the second cavity portion 186b, a second filter coating 196b is disposed on an upper surface 197 of the emitter package 171 above the second cavity portion 186b, and a third filter coating 196c is disposed on an upper surface 197 of the emitter package above the third cavity portion 186c.

Although various embodiments of the emitter package 171 are shown in FIGS. 4-17, it should be understood that other embodiments with different combinations of optical isolation structures within the cavity and filter coatings on an upper surface of the emitter package 171 are contemplated by the present disclosure. Regardless of the particular arrangement of the emitter package 171, the emitter package 171 is assembled on a skin contacting surface 166 of the computing device 100 as part of the optical sensor 170 that is designed to emit light and then measure the intensity level of one or more returned light signals to determine information about one or more parameters such as a biometric or health-related parameter.

FIG. 18 illustrates an example block diagram of such a computing device 100 according to one or more example embodiments of the disclosure. In FIG. 18, the computing device 100 includes one or more processors 150, control circuitry 151, one or more memory devices 160, one or more sensors 170 (e.g., optical sensors having a skin autofluorescence portion and a PPG portion) that include one or more emitter packages 171, one or more light detectors 172, one or more light blocking materials 174, one or more external optical filters 176, (e.g., the components of the skin autofluorescence sensors and PPG sensors discussed above), and a user interface 180.

For example, the one or more processors 150 can be any suitable processing device that can be included in a wearable computing device 100. For example, such a processor 150 may include one or more of a processor, processor cores, a controller and an arithmetic logic unit, a central processing unit (CPU), a graphics processing unit (GPU), a digital signal processor (DSP), an image processor, a microcomputer, a field programmable array, a programmable logic unit, an application-specific integrated circuit (ASIC), a microprocessor, a microcontroller, etc., and combinations thereof, including any other device capable of responding to and executing instructions in a defined manner. The one or more processors 150 can be a single processor or a plurality of processors that are operatively connected, for example in parallel. Further, the one or more processors 150 can implement control circuitry 151 which can, inter alia, be configured to reverse bias the second die 190 and/or the third die 192 while the first die 188 is emitting an emitted light signal, reverse bias the first die 188 and/or the third die 192 while the second die 190 is emitting an emitted light signal, and/or reverse bias the first die 188 and the second die 190 while the third die 192 is emitting an emitted light signal in order to avoid tainting or mixing of light signals emitted in response exposure of the dies not being used to the emitted light signal being emitted from the die in the emitter package 171 that is being used to emit a particular light signal.

The one or more memory devices 160 can include one or more non-transitory computer-readable storage mediums, such as such as a Read Only Memory (ROM), Programmable Read Only Memory (PROM), Erasable Programmable Read Only Memory (EPROM), and flash memory, a USB drive, a volatile memory device such as a Random Access Memory (RAM), a hard disk, floppy disks, a blue-ray disk, or optical media such as CD ROM discs and DVDs, and combinations thereof. However, examples of the one or more memory devices 160 are not limited to the above description, and the one or more memory devices 160 may be realized by other various devices and structures as would be understood by those skilled in the art.

The one or more memory devices 160 can include data 162 and instructions 164 that can be retrieved, manipulated, created, or stored by the one or more processor(s) 150.

In FIG. 18, the computing device 100 includes a user interface 180 configured to receive an input from a user (e.g., via a touch input such as a thumb, finger, or an input device such as a stylus or pen) or from any of the sensors 170 (e.g., data associated with the intensity levels of the returned light signals). The computing device 100 may execute a function in response to receiving the input from the user (e.g., checking health information about the user such as a blood pressure, making and/or receiving a phone call, sending and/or receiving a text message, obtaining a current time, setting a timer, a stopwatch function, controlling an external device such as a home appliance, and the like) or from any of the sensors 170 (e.g., data associated with the intensity levels of the returned light signals).

Referring still to FIG. 18, the user interface 180 includes the display 182 which displays information viewable by the user (e.g., time, date, biometric information, notifications, etc.). For example, the display 182 may be a non-touch sensitive display or a touch-sensitive display. The display 182 may include a liquid crystal display (LCD), a light emitting diode (LED) display, an organic light emitting diode (OLED) display, active matrix organic light emitting diode (AMOLED), flexible display, 3D display, a plasma display panel (PDP), a cathode ray tube (CRT) display, and the like, for example. However, the disclosure is not limited to these example displays and may include other types of displays. The display 182 may have a square or rectangular shape, or may be annular in shape (e.g., elliptical, circular, etc.). However, the shape of the display 182 is not limited thereto.

The user interface 180 may additionally, or alternatively, include one or more buttons 184 to receive an input from a user by the user applying a force to the button 184. The button 184 may be included on one or more peripheral sides of the computing device 100 as shown in FIG. 1, for example. The button 184 may include mechanical components and/or electrical circuitry to implement a function of the computing device 100 (e.g., setting a time, changing a setting and/or view of the display 182, selecting an option displayed on the display 182).

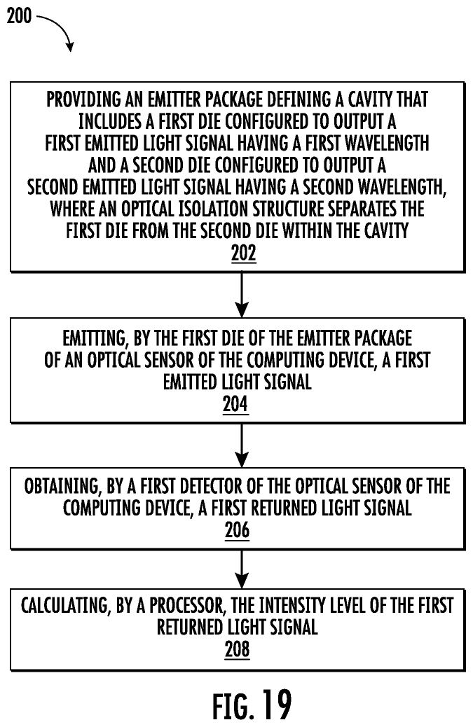

Referring now to FIG. 19, a flow diagram of a method 200 for measuring an intensity level of at least a first returned light signal via a computing device is provided. The method 200 may be implemented using, for instance, the processors 150, control circuitry 151, memory 160, data 162, and/or instructions 164 discussed above with reference to FIG. 18. FIG. 19 depicts steps performed in a particular order for purposes of illustration and discussion. Those of ordinary skill in the art, using the disclosures provided herein, will understand that various steps of the method 200 may be adapted, modified, rearranged, performed simultaneously or modified in various ways without deviating from the scope of the present disclosure.

At (202), the method 200 includes providing an emitter package defining a cavity that includes a first die configured to output a first emitted light signal having a first wavelength and a second die configured to output a second emitted light signal having a second wavelength, wherein an optical isolation structure separates the first die from the second die within the cavity.

At (204), the method 200 includes emitting, by the first die of the emitter package of an optical sensor of the computing device, the first emitted light signal.

At (206), the method 200 includes obtaining, by a first detector of the optical sensor of the computing device, the first returned light signal.

At (208), the method 200 includes calculating, by a processor, the intensity level of the first returned light signal.

In summary, the present disclosure contemplates the use of various mechanisms either alone or in combination (e.g., internal optical isolation structures within an emitter package, one or more filter coatings on an upper surface of the emitter package, and/or reverse biasing of dies in the emitter package that are not the die that is emitting the emitted light signal) to enhance the spectral purity of a returned light signal detected by a light detector and originating from a light signal emitted from a particular die within the emitter package. In other words, the light detector receives a particular returned light signal that is not contaminated with, tainted by, or mixed with returned light signals from unintended emitted light signals from other dies (e.g., light signals emitted by other dies in the emitter package) as a result of “cross-talk” between the emitted light signal from the die of interest and the other dies within the emitter package, leading to more accurate and useful returned light signal intensity data.

Aspects of the above-described example embodiments may be recorded in non-transitory computer-readable media including program instructions to implement various operations embodied by a computer. The media may also include, alone or in combination with the program instructions, data files, data structures, and the like. Examples of non-transitory computer-readable media include magnetic media such as hard disks, floppy disks, and magnetic tape; optical media such as CD ROM disks, Blue-Ray disks, and DVDs; magneto-optical media such as optical discs; and other hardware devices that are specially configured to store and perform program instructions, such as semiconductor memory, read-only memory (ROM), random access memory (RAM), flash memory, USB memory, and the like. Examples of program instructions include both machine code, such as produced by a compiler, and files containing higher level code that may be executed by the computer using an interpreter. The program instructions may be executed by one or more processors. The described hardware devices may be configured to act as one or more software modules in order to perform the operations of the above-described embodiments, or vice versa. In addition, a non-transitory computer-readable storage medium may be distributed among computer systems connected through a network and computer-readable codes or program instructions may be stored and executed in a decentralized manner. In addition, the non-transitory computer-readable storage media may also be embodied in at least one application specific integrated circuit (ASIC) or Field Programmable Gate Array (FPGA).

Each block of the flowchart illustrations may represent a unit, module, segment, or portion of code, which comprises one or more executable instructions for implementing the specified logical function(s). It should also be noted that in some alternative implementations, the functions noted in the blocks may occur out of order. For example, two blocks shown in succession may in fact be executed substantially concurrently (simultaneously) or the blocks may sometimes be executed in the reverse order, depending upon the functionality involved.

While the disclosure has been described with respect to various example embodiments, each example is provided by way of explanation, not limitation of the disclosure. Those skilled in the art, upon attaining an understanding of the foregoing, can readily produce alterations to, variations of, and equivalents to such embodiments. Accordingly, the disclosure does not preclude inclusion of such modifications, variations and/or additions to the disclosed subject matter as would be readily apparent to one of ordinary skill in the art. For example, features illustrated or described as part of one embodiment can be used with another embodiment to yield a still further embodiment. Thus, it is intended that the disclosure covers such alterations, variations, and equivalents.

What is claimed is:

1. A computing device for measuring an intensity level of at least a first returned light signal, the computing device comprising:
an optical sensor comprising:
(i) an emitter package defining a cavity, the cavity including a first die configured to output a first emitted light signal having a first wavelength and a second die configured to output a second emitted light signal having a second wavelength, wherein an optical isolation structure separates the first die from the second die within the cavity; and
(ii) a first detector configured to receive the first returned light signal; and
a processor configured to determine the intensity level of the first returned light signal.

2. The computing device of claim 1, wherein a filter coating is disposed on an upper surface of the emitter package above the first die, the second die, or both.

3. The computing device of claim 1, wherein the computing device comprises control circuitry configured to reverse bias the second die while the first die is emitting the first emitted light signal and/or to reverse bias the first die while the second die is emitting the second emitted light signal.

4. The computing device of claim 1, wherein the optical sensor further comprises a second detector configured to receive at least a second returned light signal.

5. The computing device of claim 4, wherein an optical filter blocks a portion of the first returned light signal from reaching the first detector, blocks a portion of the second returned light signal from reaching the second detector, or both.

6. The computing device of claim 5, wherein the optical filter is a long pass filter that prevents light having a wavelength that is equal to the first wavelength from reaching the first detector, prevents light having a wavelength that is equal to the second wavelength from reaching the second detector, or both.

7. The computing device of claim 5, wherein the processor is configured to calculate a skin autofluorescence level based on a measured intensity level of the first returned light signal and a measured intensity level of the second returned light signal.

8. The computing device of claim 1, wherein the first wavelength, the second wavelength, or both range from about 250 nanometers to about 900 nanometers.

9. The computing device of claim 8, wherein the first wavelength, the second wavelength, or both range from about 275 nanometers to about 500 nanometers.

10. The computing device of claim 4, wherein a light blocking material is disposed between the emitter package and the first detector, the second detector, or both.

11. The computing device of claim 1, wherein the computing device is a wearable computing device and the optical sensor is in contact with a user's skin.

12. A method for measuring an intensity level of at least a first returned light signal via a computing device, the method comprising:
providing an emitter package defining a cavity, the cavity including a first die configured to output a first emitted light signal having a first wavelength and a second die configured to output a second emitted light signal having a second wavelength, wherein an optical isolation structure separates the first die from the second die within the cavity;
emitting, by the first die of the emitter package of an optical sensor of the computing device, the first emitted light signal;
obtaining, by a first detector of the optical sensor of the computing device, the first returned light signal; and
calculating, by a processor, the intensity level of the first returned light signal.

13. The method of claim 12, wherein a filter coating is disposed on an upper surface of the emitter package above the first die, the second die, or both.

14. The method of claim 12, further comprising applying, via control circuitry, a reverse bias to the second die while the first die is emitting the first emitted light signal and/or to the first die while the second die is emitting the second emitted light signal.

15. The method of claim 12, further comprising obtaining, by a second detector of the optical sensor of the computing device, at least a second returned light signal.

16. The method of claim 15, further comprising blocking, via an optical filter, a portion of the first returned light signal from reaching the first detector, a portion of the second returned light signal from reaching the second detector, or both.

17. The method of claim 16, wherein the optical filter is a long pass filter that prevents light having a wavelength that is equal to the first wavelength from reaching the first detector, prevents light having a wavelength that is equal to the second wavelength from reaching the second detector, or both.

18. The method of claim 16, further comprising calculating a skin autofluorescence level based on a measured intensity level of the first returned light signal and a measured intensity level of the second returned light signal.

19. The method of claim 12, wherein the first wavelength, the second wavelength, or both range from about 250 nanometers to about 900 nanometers.

20. The method of claim 12, wherein the computing device is a wearable computing device and the optical sensor is in contact with a user's skin.
